(19) United States
(12) Patent Application Publication
(10) Pub. No.: US 2010/0174151 A1
(43) Pub. Date: Jul. 8, 2010

(54) MODULAR EXPANDABLE MULTIFUNCTION MACHINE WITH AUTOMATIC RECOGNITION OF ACTUATORS FOR AESTHETIC AND/OR REHABILITATIVE TREATMENTS

(76) Inventor: Francesco Peluso, Salerno (IT)

Correspondence Address: MAYER & WILLIAMS PC, 251 NORTH AVENUE WEST, 2ND FLOOR, WESTFIELD, NJ, 07090, US

(21) Appl. No.: 12/294,890

(22) PCT Filed: Mar. 27, 2006
(86) PCT No.: PCT/IT2006/000192

§ 371 Date: Sep. 26, 2008

(51) Int. Cl. A61B 5/00

(52) U.S. Cl. 600/301

(57) ABSTRACT

Modular expandable multifunction machine with automatic recognition of actuators for aesthetic and/or rehabilitative treatments. Object of the present invention is a modular, expandable machine, multifunction for the aesthetic and/or rehabilitative treatment that thanks to his own subsystem firmware, with the realization of complex architectures to the inside of the single cards and to the logical present software on board machine (so that to allow the interchange of information on the bus of system and the corrected operation), is in a position to executing multiple treatments simply connecting the appropriate applicator for the specific treatment.

### TECHNICAL FIELD

Object of the present invention is a modular, expandable machine, multifunction for the aesthetic and/or rehabilitative treatment that thanks to his own subsystem firmware, with the realization of complex architectures to the inside of the single cards and to the logical present software on board machine (so that to allow the interchange of information on the bus of system and the corrected operation), is in a position to executing multiple treatments simply connecting the appropriate applicator for the specific treatment.

### STATE OF THE TECHNIQUE

As it is know in the aesthetic centers are performed numerous types of treatments, which the definitive depilation, the electrotherapy, the mechanic peeling, the ultrasounds therapy, ionophoresis, magnetotherapy, treatments with the laser, mechanical vibration, thickening, etc. Usually an aesthetical center is subdivided in “boxes”, which are predisposed for one or more treatments according to machines that are present in the same box. Once that the customer has entered, the assistant supplies to execute the treatment demanded with the machine adapted. Obviously all the boxes cannot execute the same treatment less than every box is not equipped of all machines adapted to execute each of the treatments. Generally, every kind customer obtain a treatment for time to second of the availability of the present equipments in the box and most times the apparatus for the other treatment, demanded at the moment, is forced to move itself in an other box in how much, the machine is not present, or is absent or occupied the expert operator with that machine in order to execute the treatment. Are know some machines that have the possibility to execute also two principles of treatment at the same time (as an example electrotherapy with ultrasounds therapy), but no machine that concurs to execute different treatments (both aesthetic and therapeutic), in contemporary or independently and above all different for principle (as an example, a treatment of electrotherapy and depilation).

### SCOPES AND ADVANTAGES OF THE INVENTION

Would be therefore favourable and a great advantage the presence of an only machine in the box treatment, so that to have the availability of a variety of aesthetic-therapeutic treatments concentrates in the same place.

Scope of the present invention is therefore that to realize a machine multi function, so that rather than to use different equipment in order to execute the different treatments, with a single machine, changing the relative actuators or applicators, it is possible to execute the different typologies of aesthetic treatment.

Other scope of the present invention and that to realize a machine which automatically recognizes the applicators connected with it according to the treatment and automatically launches the management program of the treatment of the applicator itself; you have only to plug one of the applicators in a special plug and the machine recognizes it and automatically sets up by a special procedure, the program for the correct functioning of the applicator to be used by the operator who will carry out the treatment; it is not necessary that the operator programs the machine for that treatment because the machine automatically selects that kind of treatment by its control panel.

Other scope of the present invention is that one to realize a modular machine in which every module inserted in the system base is independent and able to recognize and to manage one or more applicators at the same time and independently, so that to concur with the operator at the same time to execute two or more different treatments among them.

Other scope of the present invention is that one to realize a machine supply of a procedure of self diagnostic for burlaps attendance in malfunctioning case or the remote updating from of the system of its members; in practical the user connects the appropriate cable to the taken of the telephone, and with the integrated modern interface with the system of the Company and concurs the transmission of data in center, to this stage, it is decided if to send a technician for the repair or other.

Other scope of the present invention is that to realize a modular and expandable machine with further modules, also external, for particular treatments or applications developed from third parts, where it could be necessary to have of the electronic components, specific electrical workers and/or mechanics, as in the case of pumps for the empty one for the micro derm abrasion, the everything commanded from the same logic of command.

Other scope of the present invention is that to realize a machine predisposed with a multi management system: with the system pay for use, that is the payment based on the use of the same machine paying the time of use, or by means of a canon prefixed, or paying based on the fee treatment that is being executed.

### LIST OF THE DRAWINGS AND DESCRIPTION OF THE SYSTEM

The machine object of the present invention, described in its greater generality, is composed from a main control system and from modules of expansion that connect to affixed actuators and detectors concur to execute the aesthetic/rehabilitative treatments and of monitoring the physical parameters of the human body of the customer. The main body of machine, physically is constituted from a display touch screen, a CPU, a local power source and further parts of electronics, and from the spaces predisposed for the lodging of the modules to which the actuators for the execution of the aesthetic treatments are connected. Saying machine base is ulteriorly expandable with further external modules of expansion in order to increase of necessary electronic and mechanical parts to the execution of particular aesthetic treatments where this is necessary.

FIG. 1 shows the various members of the system machine second an outline to blocks.

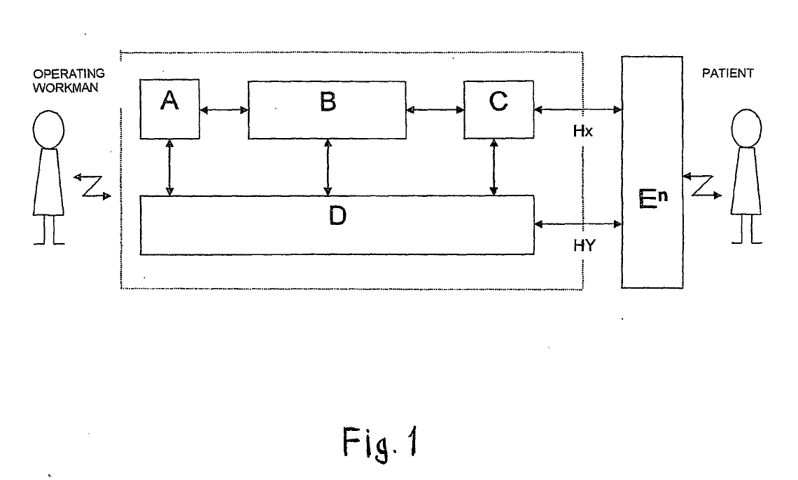

FIG. 2 shows, with an outline to blocks, the various constituent modules the machine (hardware)

FIG. 3 shows with an outline to blocks the organization of firmware.

FIG. 4 shows with an outline to blocks the organization of the software.

The system machine object of the present invention is composed of an apparatus modular (hardware) with external removable and interfaceables devices, of an distributed firmware system and from one collection of software implemented in the single modules.

### Organization of the Hardware.

The hardware is of modular type, and is essentially constituted from a panel touch-screen, from the body expandable machine, to which to go both additional modules and the terminals of the various applicators and the cable of feeding, within which the CPU, the card (or cards set) entry-out and the card (or a whole cards) of feeding are positioned.

Every module or part is connected to the CPU with which it exchanges data and information in bidirectional way; said information or data, travel on a data-bus and each component of the system is independent and in a position to executing one self diagnosis, and therefore become independent once that have received the information from the CPU. The panel touch-screen (module A) represents the interface user who concurs in contemporary is the visualization of information and parameters, than the command of functions; a series of eventual push-buttons and grip handles positioning on the same panel concurs to set up the various programs and to modify of the functionalities. Through this touch screen the exchange of the information between the user and the machine is possible. The module touch screen, composed from a panel video LCD and the appropriate film of touch screen, concurs is the visualization of the information of the machine that the relative pilotage. The touch can also visualization of relative information related to as executing the treatments, or is of the true and own images or films of “instruction” can concur also on the execution of the single treatments. The touch screen will have different dimensions to second of the machine model.

The Local power source (L block), captures the current from the net electrical worker and supplies to distribute it (by means of the appropriate bus) each to the assemblies of the apparatus after to have purposely leaked it and purposely determine the proportions based on the specifications of every single module, both the logical part and to the part of performance of the modules, let alone to relative applicators. The L block is composed from a stabilized electronic local power source that the electronic cards, concur of feeding to different tensions, all that compose the system. It is endowed with appropriate protections against the excess electrical current both in entry that in running phase of distribution or tensions to the patient. The local power source supplies beyond to the feedings standard for the operation of the system (5V, 3.3V, 12V) the feedings for driver of the actuators or applicators and the feedings for the generators of the therapy currents. It is of class of security 1 and satisfies norms IEC 601-1. The input it is to 100-220V+/−10 to 50/60 Hz. The block elaboration and control, or mother card or CPU, (B module) is constituted gives: from one CPU, one Flash memory, one memory RAM, and from controllers for the interfaces with outside devices (LCD, Touch screen, Ethernet, COM, USB, etc.) On the card it turns sw application for the management of the user interface, of the communication with the cards controller and of the communication with the outside. The communication with the Controller cards and the reader of Smart Card is of Bus type and uses a proprietary protocol. The task of block CPU is, therefore, that to manage the entire apparatus; CPU module controls the data flow among the different modules and allows both the logical and the power functions, the memorization of the machine set up parameters, the set up programs and protocols, and the different parameters which can be also set up by the user; within the memory connected to the CPU, which can be removed and expanded, besides the application program, the data of the basic therapy programs are also stored.

In this memory (removable and expandable) the formulations of the machine are all, for which in change case machine, will be sufficient to replace the memory and all data will be restored in the new equipment. There are further ulterior devices of mass memory in order to record (Data base) the various data of the patients, of various programs of treatment, of the formulations personalized, etc. The CPU concurs moreover of interfacing the machine base with eventual modules added, for evolved treatments where it is necessary one specific electronic for that treatment (example for the pressure therapy is necessary the pump for the bags, or for the micro-derm-abrasion the opportune vacuum pumps for the shot blasting are necessary). The blocks entry-out (blocks En and F) interface with block CPU and the Feeding block concurs the execution of the treatments on the patient, the generation of logical signals and of power for the transmission to the module of interface I/O beyond that to treat the information found from the patient by means of affixed sensors or detectors. Everyone of sayings modules, therefore, is equipped of an own logic that concurs to receive the information from part of the CPU and to execute them in independent way, beyond that to recognize the various actuators or applicators connects of time in time. All the actuators have a univocal code (in practical are endowed with an own inside logic with memory RAM and ROM and that it communicates with the module to serial appropriate means or with device to R-fid technology) that allow their recognition from part of the system without some programming from part of the user concurs. Practical is the device that interface with the patient to means of the different “actuators” that come connected.

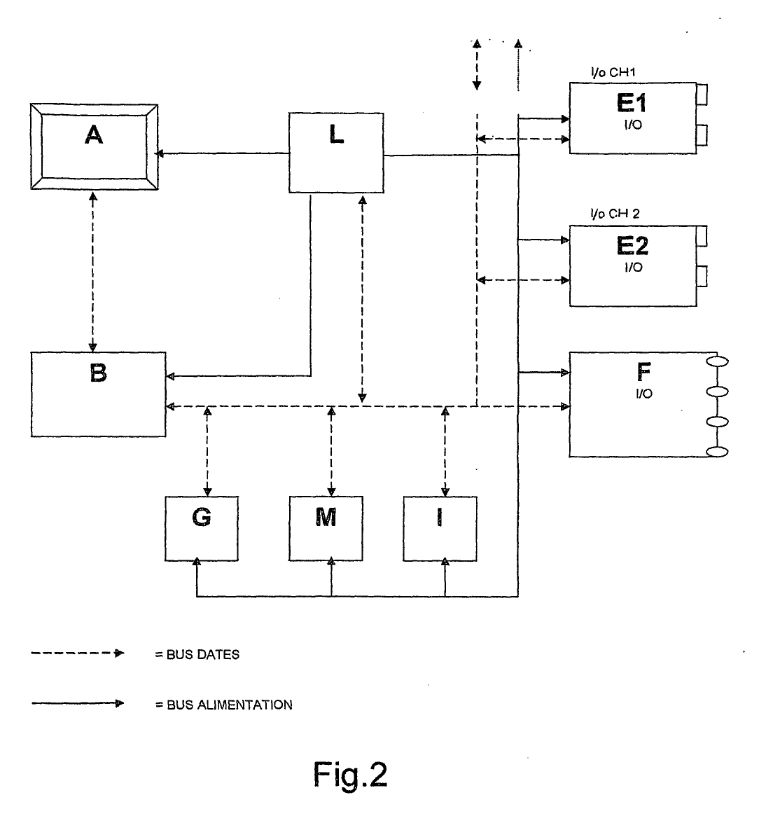

In FIG. 2 are represents the modules E1, E2, up to En, which are modules physically placed within the basic unit allowing to carry out certain kinds of treatments by simply modulating the special both logical and current information, are represented; the module F, on the contrary, allows to carry out other treatments and unlike modules E, it is installed externally to the machine and greatly augments the electronic and mechanic parts of the machine. Modules G, M, I represent further electronic parts (and they are an internal pump, a smart card reader, and audio device), which complete the block pattern of the hardware architecture.

Each expansion module consists of an electronic card allowing the recognition of the actuators and their interface to the power module for the output of the suitably modulated currents, while in some cases it allows the connection of the actuators with the CPU block for the signal processing (for example in the case of special detectors for reading the skin parameters).

The electronic card block I/O consists of:

special microcontroller, with Flash integrated memory for the management of the card, of the communication towards the main CPU and interface with stricks, sensors and actuators;
plugged up RAM memory for algorithm memorization for the generation of therapy currents, which can be updated from the main card;
digital to Analogical converters with 1024 bit resolution for the exact reproduction of the wave shapes of the generated currents;
amplifiers and drivers for the generation of the therapy currents and for the different stricks;
system of automatic recognition of the strick inserted in the interface with direct communication with the application in the CPU;
protection with alarm generation for over-currents and over-voltages.

### Organization of Firmware System

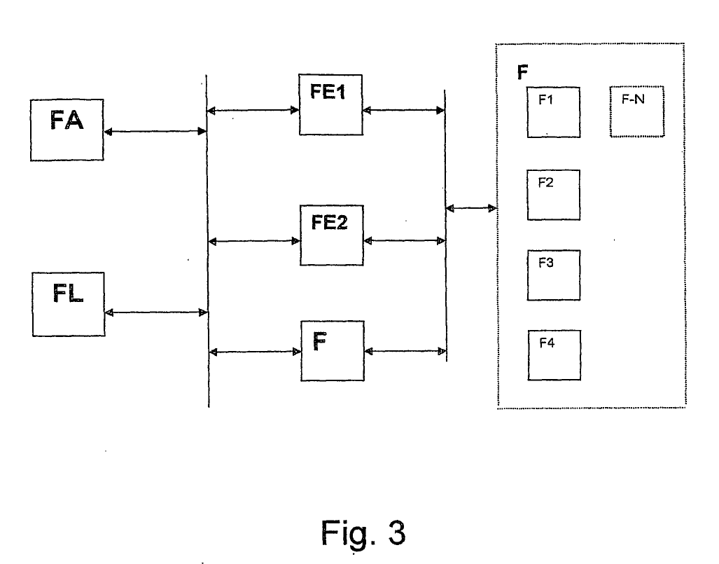

In FIG. 3 is represented the organization of the firmware system implemented on board of various assemblies of the machine system object of the present invention. Every electronic card that composes the system is equipped of one own logic of control and consequently also of one own “identity and functionality”. The task of firmware (F) of which every device is endowed is that to contain to its inside the essential parameters of coding of the data bus of system, that is to be in a position to identifying in correct way the information that originate from the CPU and from the others members of the system and then to have to its inside an appropriate location of memory where come records, in factory, the greater part of the parameters works them necessary to the self diagnostic of the card, in such way from being able to always come to an agreement the system to the maximum functionality, (in reference to the 1° level, while the 2° level is updating directly from remote or with the rows of update loaded to appropriate means procedure).

The CPU itself is provided for its own firmware (Fa) allowing the functioning of the whole architecture of the machine and contains the information for starting the machine, for the loading from the ROM external devices, for the automatic loading of the Operative System Software and then for the further software; within this firmware the software tracks related to the interface and the data bus control, and a stack providing for communications with the further expansions, are included.

On board of the external ROM to the processor also an firmware appropriate is implemented. The processor on board of I/O card is equipped of own firmware (Fen) and (Ff), also in this case it is possible to install it also on one external ROM. Present firmware, concurs with I/O card of interface with the CPU and the actuators, identifying the type of actuator that comes to it connected or the type of detector of parameters that comes to it connected. Present firmware on board I/O card, therefore, concurs the operation of I/O card in totally independent way regarding the central CPU, having in its inside memorized all the parameters of operation sets up from the operator. Present firmware on board of the Feeding card (FI) concurs of talk with the remaining parts of the machine, in reference to the distribution of electrical current, to the electrical current overloaded, the self diagnostic. Firmware concurs also to control the electrical dispersions of the system in reference to the security of the patient. The system therefore is in a position to verifying also the optimizations of the electrical consumptions. Firmware (Fn) present in every actuator or the detector, accredited for the operation with the machine, beyond identifying them and therefore to concur their acknowledgment from part of the machine, is also in a position to containing a series of own parameters of the applicator, pre-loaded in factory or updating of time in time from the same machine. Therefore every single actuator is equipped of an appropriate firmware that it contains the identification code of the applicator, the parameters for the corrected operation, a memory location available for the loading of the functioning hours and the personal codification by the user, are registered.

It is then possible to have a feedback between the data present on the actuator and those sensed by the machine, in order also to check the integrity of the actuator itself, for a prosecution of the treatment of for replacing the applicator resulting to be not conforming with the minimum safety rules.

### Organization of the Software System.

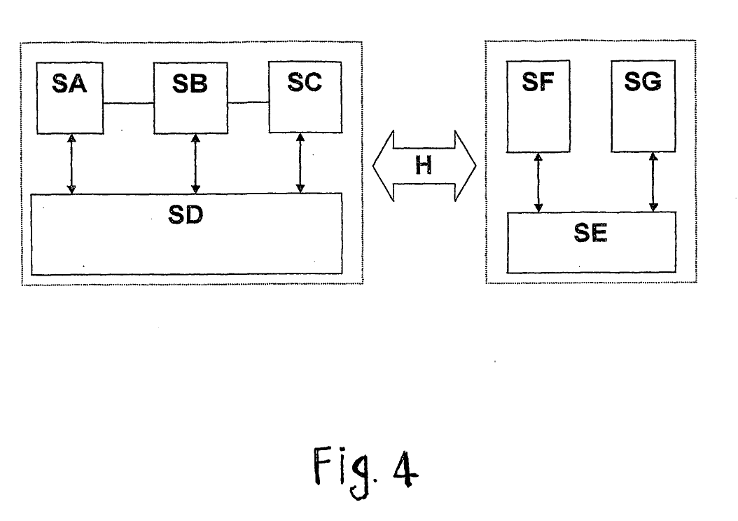

The system object of the present invention is equipped of just a software of operation that concurs the management and the eventual modification of the detailed lists of the reported treatments with the single patients (of which has the database), and the visualization of the course for like executing the treatment. The software on board machine is updating at a distance to means an appropriate procedure of upgrade. FIG. 4 shows second an outline to blocks, the organization of the software distinguished in two affixed programs, that implemented on the hardware of the system (Sd) and that on external PC (Se).

The software (Sd) implemented on the hardware allows the management on three macro areas: of the display and the applications (Sa), the control of the system parameters and of the hardware sets, also for tele-assistance and control (Sb), and the updating subsystem of the specific functions for each standard applicator or for each new applicator which can be interfaced with the machine (Sc).

The operating system comes loaded, in factory, directly on board machine, on the CPU, it concurs the loading of the application program purposely realized that the execution of the treatments is the inherent motor. The functions more important are: control and acknowledgment of the actuator or detector, modification of the parameters standard of a treatment protocol, procedure for upgrade of the new applicators or new versions software and firmware, procedure of self diagnostic of the hardware, procedure for the connection from and for remote, management of the protocols of communication with the various modules, updating with new treatments and modification of those already sets up with eventual rename, management of the supports of memory in reference to the treatments is executed and to execute on the patient, program of safety and management of the procedure pay for use, the maintenance programs, press with printing connected (or to means radio or with specific cable) and elaboration of treatment to execute in reference to data finds with the detectors. The software on PC is a limited version of the system on board of the machine (and it is not closely necessary for the operation of the machine), and concurs (Sf block) to connect themselves with the general site of the company and to receive upgrade the software for the following passage on board machine. Moreover the software (Sg) has the function of being able to manage also (activating appropriate functionalities of the procedure) the user, a minimum of accounting and inherent management the activities executed with the machine. In fact, unloading from web site or CD, the rows of update and connected the machine (or transferred the rows to average SD, or memory device USB) it is possible to execute the updating of the machine. One of the special and innovative characteristics of the system is represented therefore from its expandability and modularity. In fact, it is possible to widen the number of executable treatments at the same time adding ulterior modules on board.

The typology of modules to be added varies according to the treatment typology needed. Two families of modules for the expansion of the system exist therefore. One first family is represented from multi-treatment modules are added to control different applicators or detectors, that is they can carry out different treatment typologies at the same time, which are different from each other such as thermal and ultrasound therapies, electrotherapies, brushes, etc., or modules which can carry out only one treatment are added, or modules are added when it is necessary to augment the hardware, with special mechanic parts or special electronic sets necessary for a specific treatment (in practical in order executing, as an example, the treatment of pressure therapy, they need a series of pumps, and therefore, not having these pumps on board of the machine, it is necessary to increase to the configuration hardware of the machine with an appropriate module that contains to its inside, beyond the card electronic, also these ulterior pumps with the adequate connector), in kind this type of expansions settles to the outside of the module base. Other particular innovation is that, changing the applicator to the machine, automatically the program for the operation of the new actuator is recalled. It is not possible, therefore, that the operator selections an execution program treatment with a wrong actuator, in how much this type of selection happens in automatic rifle.

The logical recognition and use signals of applicators can be sent and read by the R-fid technology too, beyond that on serial device. In some cases when the same applicator is in a position to carrying out different treatments, the software concurs with the operator to choose and to select the type of treatment to use between the different proposals and executable with that type of applicator. The machine moreover, is in a position to being connected also to some external modules o machines produced from third parts and that have the adequate interface. In this category they can re-enter as an example solar therapy small bed, or of loss of weight, solarium box, small bed for magneto therapy, etc. . . . . The applicators of treatment and the detectors of data of the patient (the detectors in a position to reading the parameters of the skin like the photo type, hydration, elasticity, sebo, PH, mass, etc.) connected to the machine are multiple (can use everyone a various principle of operation, mechanic, ultrasounds, infrared, laser, stimulation through electrical currents workers and magnetism, luminous frequencies, etc.) and they space between those adapted to the treatment of cleaning and stimulation of the face, depilation, to those applicators for the therapeutic and aesthetic treatment of the human body.

1) Expandable modular machine for aesthetic/rehabilitative treatments made of a modular hardware apparatus essentially consisting of a touch-screen panel, of the expandable body of the machine, within which the CPU, the input-output card (or a set of cards) and the feeding card (or a set of cards) are placed, of a distributed firmware and of a collection of software implemented in the single modules, to which both additional modules and the terminals of the different applicators and the feeding cable are connected characterized by the following: each module or part of it is connected to the CPU with which it exchanges two-way data and information and such information or data, travel on a data bus; each component of the system, I/O blocks included, provided with their own processor, once they have received the information from the central CPU, become independent and are able to carry out a self-diagnosis; I/O blocks interface with the treatment actuators or the detector of the patient's parameters, they univocally identify and control them in the carrying out of one or more specific treatments also at the same time and independently from each other.

2) Expandable modular machine for aesthetic/rehabilitative treatments as claimed in 1) characterized by the fact that the electronic card block I/O consists of: special microcontroller, with Flash integrated memory for the management of the card, of the communication towards the main CPU and interface with stricks, sensors and actuators; plugged up RAM memory for algorithm memorization for the generation of therapy currents, which can be updated from the main card; digital to analogical converters with 1024 bit resolution for the exact reproduction of the wave shapes of the generated currents; amplifiers and drivers for the generation of the therapy currents and for the different stricks; system of automatic recognition of the strick inserted in the interface with direct communication with the application in the CPU; protection with alarm generation for over-currents and over-voltages.

3) Expandable modular machine for aesthetic/rehabilitative treatments as claimed in 1) characterized by the fact that the CPU, (module B) consists of a processor, of a Flash memory, of a RAM memory, and of controllers for the interfaces with the outside, of a LCD, a touch-screen, Ethernet, COM; USB etc., and the CPU itself is provided for its own firmware (Fa) allowing the functioning of the whole architecture of the machine and contains the information for starting the machine, for the loading from the ROM external devices, for the automatic loading of the Operative System Software and then for the further software; within this firmware the software tracks related to the interface and the data bus control, and a stack providing for communications with the further expansions, are included.

4) Expandable modular machine for aesthetic/rehabilitative treatments as claimed in 1) characterized by the fact that the CPU module controls the data flow among the different modules and allows both the logical and the power functions, the memorization of the machine set up parameters, the set up programs and protocols, and the different parameters which can be also set up by the user, within the memory connected to the CPU, which can be removed and expanded, besides the application program, the data of the basic therapy programs are also stored.

5) Expandable modular machine for aesthetic/rehabilitative treatments as claimed in 1) characterized by the fact that there are a lot of treatment applicators and detectors of the patient's data, which can be connected to the machine, each of them can resort to a different functioning mode, such as the mechanic, ultrasound, infrared, laser, functioning and the functioning by stimulation by electric currents and magnetism light frequencies etc., and they cover a wide range of applicators, from those used for face cleansing and stimulation and depilation treatments to those used for the body therapeutic and aesthetic treatments.

6) Expandable modular machine for aesthetic/rehabilitative treatments as claimed in 1) characterized by the fact that the logical recognition and use signals of applicators can be sent and read by the R-fid technology too.

7) Expandable modular machine for aesthetic/rehabilitative treatments as claimed in 1) characterized by the fact that the typology of modules to be added varies according to the treatment typology needed: multi-treatment modules are added to control different applicators or detectors, that is they can carry out different treatment typologies at the same time, which are different from each other such as thermal and ultrasound therapies, electrotherapies, brushes, etc., or modules which can carry out only one treatment are added, or modules are added when it is necessary to augment the hardware, with special mechanic parts or special electronic sets necessary for a specific treatment.

8) Expandable modular machine for aesthetic/rehabilitative treatments as claimed in 1) characterized by the fact that the software (Sd) implemented on the hardware allows the management of the display and the applications (Se), the control of the system parameters and of the hardware sets, also for tele-assistance and control (Sb), and the updating subsystem of the specific functions for each standard applicator or for each new applicator which can be interfaced with the machine (Sc).

9) Expandable modular machine for aesthetic/rehabilitative treatments as claimed in 1) characterized by the fact that the machine is preset with a pay per use system, that is payment according to the time amount of use of the machine itself, or payment according to the treatment which is in course.

10) Expandable modular machine for aesthetic/rehabilitative treatments as claimed in 1) characterized by the fact that inside each actuator the essential codification parameters of the data bus of the system are contained, so that it is possible to correctly identify the information coming from the PCU and from the other components of the system and then to have inside it a special memory location where the most part of the functional parameters necessary for the self-diagnostics of the card, the loading of the functioning hours and the personal codification by the user, are registered; it is then possible to have a feedback between the data present on the actuator and those sensed by the machine, in order also to check the integrity of the actuator itself, for a prosecution of the treatment of for replacing the applicator resulting to be not conforming with the minimum safety rules.
